(19) United States
(12) Patent Application Publication
(10) Pub. No.: US 2017/0157025 A1
(43) Pub. Date: Jun. 8, 2017

(54) TEXTILE FRIENDLY ANTIPERSPIRANT COMPOSITIONS

(71) Applicant: Henkel AG & Co. KGaA, Duesseldorf (DE)

(72) Inventor: Thomas Doering, Dormagen (DE)

(73) Assignee: Henkel AG & Co. KGaA, Duesseldorf (DE)

(21) Appl. No.: 15/371,991

(22) Filed: Dec. 7, 2016

(30) Foreign Application Priority Data
Dec. 8, 2015 (DE) 10 2015 224 559.4

(51) Int. Cl. A61K 8/891; A61K 8/37; A61K 8/28; A61K 8/86; A61Q 15/00; A61K 8/26

(52) U.S. Cl. CPC A61K 8/891; A61Q 15/00; A61K 8/26; A61K 8/28; A61K 8/86; A61K 8/37; A61K 2800/48

(57) ABSTRACT

The subject of the present invention is an antiperspirant composition for personal body care with an improved residue behavior, which includes a mixture of at least one volatile silicone oil, PPG-14 butyl ether, and at least one other oil, selected from 2-phenoxyethyl octanoate and PPG-3 myristyl ether.

### FIELD OF THE INVENTION

The present invention generally relates to substantially water-free antiperspirant compositions, which have an improved residue behavior in regard to textile staining.

### BACKGROUND OF THE INVENTION

Commercial perspiration-inhibiting compositions, also referred to as antiperspirants below, include as the active antiperspirant substance at least one water-soluble, astringent inorganic and/or organic salt of aluminum, zirconium, or selected aluminum-zirconium mixed salts. The active antiperspirant substances have no direct effect on the activity of the sweat glands but minimize sweat secretion by narrowing the discharge ducts. The Al salts inhibit perspiration on treated areas of skin by superficial blocking of the sweat gland ducts as a result of Al mucopolysaccharide deposits. Antiperspirant compositions are generally applied in the axillary areas. When the composition dries on the skin or on clothing which has come into contact with the skin after application of the antiperspirant, the antiperspirant salt often remains visible as a white residue. This also occurs, even if to a much lesser extent, with highly water-containing compositions in which the antiperspirant salt is present in dissolved form. The white residues are perceived by the user as a highly negative property of the product. For masking aluminum salt residues of water-containing compositions, both water-soluble components, such as in particular 1,2-propylene glycol, as well as oils, in particular ester oils such as isopropyl palmitate or alkyl benzoate, are known in the prior art. Masking agents of this type wet the antiperspirant salt and do not evaporate even after application to the skin, as do water and cyclomethicone, for example. As a result, the antiperspirant salt dries significantly more slowly and the occurrence of visible residues is delayed. The masking can be improved further by selecting a masking agent having a refractive index n_{D} in the range of the refractive index n_{D} of typical antiperspirant salts or typically used aqueous solutions of antiperspirant salts, therefore, in the range of n_{D}²⁰=1.4 to 1.5. Many of the oils with a high masking action have the disadvantage that they can lead to staining of the clothing (“fabric staining”), which is difficult to wash out.

Antiperspirant compositions are available in various delivery forms, for example, as a composition that can be sprayed with a propellant. Compositions of this type are usually filled into spray cans made from aluminum or (less frequently) tinplate, which are protected from corrosion by an internal coating. Despite this protective coating, however, corrosion damage can always occur. A further problem with such products is that the valve becomes blocked. A composition that inhibits corrosion and/or reduces valve blockage would therefore be desirable for these specific delivery forms.

There is a constant need, therefore, for antiperspirant compositions with reduced fiber staining, high residue masking, and reduced visible residues.

An object of the present invention was to provide antiperspirant compositions that cause the lowest possible fiber or textile staining, in particular a lowest possible wash-resistant fiber or textile staining.

A further object of the present invention was to provide antiperspirant compositions with high residue masking and reduced visible residues.

A particular challenge was the object of achieving a lowest possible fiber or textile staining, in particular a lowest possible wash-resistant fiber or textile staining, and simultaneously the best possible masking of visible residues.

Surprisingly, it has been found that the aforesaid objects are achieved by certain combinations of oil components.

Furthermore, other desirable features and characteristics of the present invention will become apparent from the subsequent detailed description of the invention and the appended claims, taken in conjunction with this background of the invention.

### BRIEF SUMMARY OF THE INVENTION

An antiperspirant composition for personal body care, including a) at least one active antiperspirant substance, which is present in suspended, undissolved form and is selected from aluminum salts and aluminum-zirconium salts; b) 0-7% by weight, preferably 0-3% by weight of free water; c) a mixture of at least three oils that are liquid under normal conditions, wherein the composition includes c1) at least one volatile silicone oil in a total amount of 28 to 61% by weight, preferably 30 to 50% by weight, particularly preferably 32 to 45% by weight, exceptionally preferably 35 to 38% by weight; furthermore c2) 7 to 20% by weight, preferably 8 to 17% by weight, particularly preferably 11 to 16% by weight, exceptionally preferably 12 to 13% by weight of PPG-14 butyl ether; furthermore c3) 0 to 18% by weight, preferably 0.1 to 15% by weight, particularly preferably 0.5 to 13% by weight, exceptionally preferably 1 to 7% by weight of 2-phenoxyethyl octanoate; and c4) 0 to 7% by weight, preferably 0.3 to 6% by weight, particularly preferably 1 to 5% by weight, exceptionally preferably 2 to 4% by weight of PPG-3 myristyl ether; with the proviso that the total amount of oils c2)+c3)+c4) is a maximum of 30% by weight, wherein all quantities given in percentage by weight in each case refer to the weight of the composition, without taking into account optionally present propellants, and with the proviso that at least one of the oils, 2-phenoxyethyl octanoate or PPG-3 myristyl ether, is included.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A subject of the present invention is an antiperspirant composition for personal body care, including
a) at least one active antiperspirant substance, which is present in suspended, undissolved form and is selected from aluminum salts and aluminum-zirconium salts,
b) 0-7% by weight, preferably 0-3% by weight of free water,
c) a mixture of at least three oils that are liquid under normal conditions,
   wherein the composition includes
   c1) at least one volatile silicone oil in a total amount of 28 to 61% by weight, preferably 30 to 50% by weight, particularly preferably 32 to 45% by weight, exceptionally preferably 35 to 38% by weight, furthermore
   c2) 7 to 20% by weight, preferably 8 to 17% by weight, particularly preferably 11 to 16% by weight, exceptionally preferably 12 to 13% by weight of PPG-14 butyl ether, furthermore
   c3) 0 to 18% by weight, preferably 0.1 to 15% by weight, particularly preferably 0.5 to 13% by weight, exceptionally preferably 1 to 7% by weight of 2-phenoxyethyl octanoate, and
   c4) 0 to 7% by weight, preferably 0.3 to 6% by weight, particularly preferably 1 to 5% by weight, exceptionally preferably 2 to 4% by weight of PPG-3 myristyl ether,
      with the proviso that the total amount of oils c2)+c3)+c4) is a maximum of 30% by weight, wherein all quantities given in percentage by weight in each case refer to the weight of the composition, without taking into account optionally present propellants, and with the proviso that at least one of the oils, 2-phenoxyethyl octanoate or PPG-3 myristyl ether, is included.

“Normal conditions” in the context of the present invention are a temperature of 20° C. and a pressure of 1013.25 mbar. Melting point data also refer to a pressure of 1013.25 mbar.

All quantitative data, unless otherwise specified, refer to the total weight of the antiperspirant composition of the invention. Any added propellants are not included in the antiperspirant composition of the invention, and therefore all quantitative data refer to the total weight of the propellant-free antiperspirant composition, unless otherwise specified.

“Free water” in the context of the present invention is water which is not included in the antiperspirant composition in the form of water of crystallization, water of hydration, or similarly molecularly bound water. The content of water of crystallization, water of hydration, or similarly molecularly bound water which is included in the components used, in particular in the active antiperspirant substances, does not represent free water in the context of the present invention. Free water is, for example, water that is added to the composition of the invention as a solvent, as a gel activator, or as a solvent component of other active substances.

The antiperspirant compositions of the invention, based on their total weight, include 0 to 7% by weight of free water. Antiperspirant compositions preferred according to the invention, based on their total weight, include 0 to 6% by weight of free water, preferably 0 to 5% by weight, particularly preferably 0 to 4% by weight, exceptionally preferably 0 to 3% by weight of free water. The antiperspirant compositions of the invention are therefore to be regarded as substantially free of water.

The compositions of the invention include at least one active antiperspirant substance, which is selected from aluminum salts and aluminum-zirconium salts. Preferred active antiperspirant substances are selected from the water-soluble, astringent inorganic and organic salts of aluminum and zirconium or any mixtures of these salts.

According to the invention, water solubility is understood to mean a solubility of at least 3% by weight at 20° C., in other words, that amounts of at least 3 g of the active antiperspirant substance are soluble in 97 g of water at 20° C. According to the invention, water solubility is preferably understood to mean a solubility of at least 5% by weight at 20° C., in other words, that amounts of at least 5 g of the active antiperspirant substance are soluble in 95 g of water at 20° C.

Particularly preferred active antiperspirant substances are selected from aluminum chlorohydrate, in particular aluminum chlorohydrate with the general formula [Al₂(OH)₅Cl.1-6H₂O]ₙ, preferably [Al₂(OH)₅Cl.2-3H₂O]ₙ, which can be present in non-activated or in activated (depolymerized) form, and aluminum chlorohydrate with the general formula [Al₂(OH)₄Cl₂.1-6H₂O]ₙ, preferably [Al₂(OH)₄Cl₂.2-3H₂O]ₙ, which can be present in non-activated or in activated (depolymerized) form.

Preferred, further, are aluminum sesquichlorohydrate, aluminum dichlorohydrate, aluminum chlorohydrex propylene glycol (PG) or aluminum chlorohydrex polyethylene glycol (PEG), aluminum or aluminum-zirconium glycol complexes, e.g., aluminum or aluminum-zirconium propylene glycol complexes, aluminum sesquichlorohydrex PG or aluminum sesquichlorohydrex PEG, aluminum PG dichlorohydrex or aluminum PEG dichlorohydrex, aluminum hydroxide, selected further from aluminum-zirconium chlorohydrates, such as aluminum-zirconium trichlorohydrate, aluminum-zirconium tetrachlorohydrate, aluminum-zirconium pentachlorohydrate, aluminum-zirconium octachlorohydrate, aluminum-zirconium chlorohydrate glycine complexes, such as aluminum-zirconium trichlorohydrex glycine, aluminum-zirconium tetrachlorohydrex glycine, aluminum-zirconium pentachlorohydrex glycine, aluminum-zirconium octachlorohydrex glycine, potassium aluminum sulfate (KAl(SO₄)₂.12H₂O, alum), dehydrated alum (KAl(SO₄)₂ with 0 to 11 moles of water of crystallization), sodium aluminum chlorohydroxylactate, aluminum bromohydrate, aluminum chloride, aluminum sulfate, aluminum lactate, sodium aluminum chlorohydroxylactate, zirconyl oxyhalides, in particular zirconyl oxychlorides, zirconyl hydroxyhalides, in particular zirconyl hydroxychlorides (zirconium chlorohydrate).

Active antiperspirant substances particularly preferred according to the invention are selected from so-called “activated” aluminum and aluminum-zirconium salts, which are also referred to as active antiperspirant substances “with enhanced activity”. Such active substances are known in the prior art and are commercially available. Activated aluminum and aluminum-zirconium salts are generally produced by heat treatment of a relatively dilute solution of the salt (e.g., about 10% by weight of salt) in order to increase its HPLC peak 4 to peak 3 area ratio. The activated salt can then be dried to a powder, in particular spray-dried. In addition to spray-drying, e.g., roller drying is also suitable.

Activated aluminum and aluminum-zirconium salts typically have an HPLC peak 4 to peak 3 area ratio of at least 0.4, preferably at least 0.7, particularly preferably at least 0.9, wherein at least 70% of the aluminum can be assigned to these peaks.

Activated aluminum and aluminum-zirconium salts do not necessarily have to be used as a spray-dried powder. Active antiperspirant substances likewise preferred according to the invention are nonaqueous solutions or solubilizates of an activated antiperspirant aluminum or aluminum-zirconium salt, which are stabilized against loss of activation against the rapid degradation of the HPLC peak 4:peak 3 area ratio of the salt by the addition of an effective amount of a polyhydric alcohol having 3 to 6 carbon atoms and 3 to 6 hydroxyl groups, preferably propylene glycol, sorbitol, and pentaerythritol. For example, compositions are preferred which include in percentage by weight (USP): 18-45% by weight of an activated aluminum or aluminum-zirconium salt, 55-82% by weight of at least one water-free polyhydric alcohol having 3 to 6 carbon atoms and 3 to 6 hydroxyl groups, preferably propylene glycol, butylene glycol, diethylene glycol, dipropylene glycol, glycerol, sorbitol, and pentaerythritol, particularly preferably propylene glycol.

Also particularly preferred are complexes of activated antiperspirant aluminum or aluminum-zirconium salts with a polyhydric alcohol, which include 20-50% by weight, particularly preferably 20-42% by weight, of activated antiperspirant aluminum or aluminum-zirconium salt and 2-16% by weight of molecularly bound water, wherein the balance to 100% by weight is at least one polyhydric alcohol having 3 to 6 carbon atoms and 3 to 6 hydroxyl groups. Propylene glycol, propylene glycol/sorbitol mixtures, and propylene glycol/pentaerythritol mixtures are preferred alcohols of this type.

Other preferred active antiperspirant substances are basic calcium-aluminum salts. These salts are produced by reacting calcium carbonate with aluminum chlorohydroxide or aluminum chloride and aluminum powder or by adding calcium chloride dihydrate to aluminum chlorohydroxide.

Other preferred active antiperspirant substances are aluminum-zirconium complexes, which are buffered with salts of amino acids, in particular with alkali and alkaline earth glycinates. Other preferred active antiperspirant substances are activated aluminum or aluminum-zirconium salts, including 5-78% by weight (USP) of an activated antiperspirant aluminum or aluminum-zirconium salt, an amino acid or hydroxyalkanoic acid in an amount so as to provide an (amino acid or hydroxyalkanoic acid) to (Al+Zr) weight ratio of 2:1-1:20 and preferably 1:1 to 1:10, and a water-soluble calcium salt in an amount so as to provide a Ca:(Al+Zr) weight ratio of 1:1-1:28 and preferably 1:2-1:25. Particularly preferred solid activated antiperspirant salt compositions include 48-78% by weight (USP), preferably 66-75% by weight, of an activated aluminum or aluminum-zirconium salt and 1-16% by weight, preferably 4-13% by weight, of molecularly bound water (water of hydration), further sufficient water-soluble calcium salt so that the Ca:(Al+Zr) weight ratio is 1:1-1:28, preferably 1:2-1:25, and sufficient amino acid so that the amino acid to (Al+Zr) weight ratio is 2:1-1:20, preferably 1:1-1:10.

Other particularly preferred solid antiperspirant activated salt compositions include 48-78% by weight (USP), preferably 66-75% by weight of an activated aluminum or aluminum-zirconium salt and 1-16% by weight, preferably 4-13% by weight of molecularly bound water (water of hydration), further sufficient water-soluble calcium salt so that the Ca:(Al+Zr) weight ratio is 1:1-1:28, preferably 1:2-1:25, and sufficient glycine so that the glycine to (Al+Zr) weight ratio is 2:1-1:20, preferably 1:1-1:10.

Other particularly preferred solid antiperspirant activated salt compositions include 48-78% by weight (USP), preferably 66-75% by weight of an activated aluminum or aluminum-zirconium salt and 1-16% by weight, preferably 4-13% by weight of molecularly bound water, further sufficient water-soluble calcium salt so that the Ca:(Al+Zr) weight ratio is 1:1-1:28, preferably 1:2-1:25, and sufficient hydroxyalkanoic acid so that the hydroxyalkanoic acid to (Al+Zr) weight ratio is 2:1-1:20, preferably 1:1-1:10.

Water-soluble calcium salts preferred for stabilizing the antiperspirant salts are selected from calcium chloride, calcium bromide, calcium nitrate, calcium citrate, calcium formate, calcium acetate, calcium gluconate, calcium ascorbate, calcium lactate, calcium glycinate, calcium carbonate, calcium sulfate, calcium hydroxide, and mixtures thereof.

Amino acids preferred for stabilizing the antiperspirant salts are selected from glycine, alanine, leucine, isoleucine, β-alanine, valine, cysteine, serine, tryptophan, phenylalanine, methionine, β-amino-n-butanoic acid, and γ-amino-n-butanoic acid, and the salts thereof, in each case in the d-form, the l-form, and the dl-form; glycine is particularly preferred.

Preferred hydroxyalkanoic acids for stabilizing the antiperspirant salts are selected from glycolic acid and lactic acid.

Other preferred active antiperspirant substances are activated aluminum or aluminum-zirconium salts, including 5-78% by weight (USP) of an activated antiperspirant aluminum or aluminum-zirconium salt, an amino acid or hydroxyalkanoic acid in an amount so as to provide an (amino acid or hydroxyalkanoic acid) to (Al+Zr) weight ratio of 2:1-1:20 and preferably 1:1 to 1:10, and a water-soluble strontium salt in an amount so as to provide a Sr:(Al+Zr) weight ratio of 1:1-1:28 and preferably 1:2-1:25.

Particularly preferred solid antiperspirant activated salt compositions include 48-78% by weight (USP), preferably 66-75% by weight of an activated aluminum or aluminum-zirconium salt, and 1-16% by weight, preferably 4-13% by weight of molecularly bound water, further, sufficient water-soluble strontium salt so that the Sr:(Al+Zr) weight ratio is 1:1-1:28, preferably 1:2-1:25, and sufficient amino acid so that the amino acid to (Al+Zr) weight ratio is 2:1-1:20, preferably 1:1-1:10.

Other particularly preferred solid antiperspirant activated salt compositions include 48-78% by weight (USP), preferably 66-75% by weight of an activated aluminum or aluminum-zirconium salt, and 1-16% by weight, preferably 4-13% by weight of molecularly bound water, further sufficient water-soluble strontium salt so that the Sr:(Al+Zr) weight ratio is 1:1-1:28, preferably 1:2-1:25, and sufficient glycine so that the glycine to (Al+Zr) weight ratio is 2:1-1:20, preferably 1:1-1:10.

Other particularly preferred solid antiperspirant activated salt compositions include 48-78% by weight (USP), preferably 66-75% by weight of an activated aluminum or aluminum-zirconium salt and 1-16% by weight, preferably 4-13% by weight of molecularly bound water, and also sufficient water-soluble strontium salt so that the Sr:(Al+Zr) weight ratio is 1:1-1:28, preferably 1:2-1:25, and sufficient hydroxyalkanoic acid so that the hydroxyalkanoic acid to (Al+Zr) weight ratio is 2:1-1:20, preferably 1:1-1:10.

Other preferred activated aluminum salts are those of the general formula Al₂(OH)₆₋ₐXa, where X is Cl, Br, I, or NO₃ and “a” is a value of 0.3 to 5, preferably of 0.8 to 2.5, and particularly preferably 1 to 2, so that the molar ratio of Al:X is 0.9:1 to 2.1:1, as disclosed, e.g., in U.S. Pat. No. 6,074,632. In these salts, some water of hydration is generally associatively bound, typically 1 to 6 moles of water per mole of salt. Particularly preferred is aluminum chlorohydrate (i.e., X is Cl in the aforementioned formula) and especially 5/6-basic aluminum chlorohydrate, where “a” is 1, so that the molar ratio of aluminum to chlorine is 1.9:1 to 2.1:1. Zirconium-free aluminum sesquichlorohydrates particularly preferred according to the invention have a molar metal to chloride ratio of 1.5:1-1.8:1.

Preferred activated aluminum-zirconium salts are those that represent mixtures or complexes of the aluminum salts described above with zirconium salts of the formula ZrO(OH)₂₋ₚₚY_{b}, where Y is Cl, Br, I, NO₃, or SO₄, b is a rational number from 0.8 to 2, and p is the valence of Y, as disclosed, e.g., in U.S. Pat. No. 6,074,632. The zirconium salts generally also have some associatively bound water of hydration, typically 1 to 7 moles of water per mole of salt. The zirconium salt is preferably zirconyl hydroxychloride with the formula ZrO(OH)_{2-b}Cl_{b}, where b is a rational number from 0.8 to 2, preferably 1.0 to 1.9. Preferred aluminum-zirconium salts have an Al:Zr molar ratio of 2 to 10 and a metal:(X+Y) ratio of 0.73 to 2.1, preferably 0.9 to 1.5. A particularly preferred salt is aluminum-zirconium chlorohydrate (i.e., X and Y are Cl), which has an Al:Zr ratio of 2 to 10 and a molar metal:Cl ratio of 0.9 to 2.1. The term aluminum-zirconium chlorohydrate comprises the tri-, tetra-, penta-, and octachlorohydrate forms.

Zirconium salts preferred according to the invention have the general formula ZrO(OH)₂₋ₐClₐ.xH₂O with a=1.5-1.87 and x=1-7, where a and x are rational numbers.

Preferred aluminum-zirconium salts have a molar metal to chloride ratio of 0.9-1.3, preferably 0.9-1.1, particularly preferably 0.9-1.0.

Preferred aluminum-zirconium chlorohydrates generally have the empirical formula AlₙZr(OH)_{[3n+4-m(n+1)}](C)_{[m(n+1)]} with n=2.0-10.0, preferably 3.0-8.0, m=0.77-1.11 (corresponding to a molar metal (Al+Zr) to chloride ratio of 1.3-0.9), preferably m=0.91-1.11 (corresponding to M:Cl=1.1-0.9), and particularly preferably m=1.00-1.11 (corresponding to M:Cl=1.0-0.9), further very preferably m=1.02-1.11 (corresponding to M:Cl=0.98-0.9), and very preferably m=1.04-1.11 (corresponding to M:Cl=0.96-0.9).

In these salts there is generally some associatively bound water of hydration, typically 1-6 moles of water per mole of salt, corresponding to 1-16% by weight, preferably 4-13% by weight of water of hydration.

The preferred aluminum-zirconium chlorohydrates are usually associated with an amino acid in order to prevent the polymerization of the zirconium species during preparation. Preferred stabilizing amino acids are selected from glycine, alanine, leucine, isoleucine, β-alanine, cysteine, valine, serine, tryptophan, phenylalanine, methionine, β-amino-n-butanoic acid, and γ-amino-n-butanoic acid and the salts thereof, in each case in the d-form, the l-form, and the dl-form; glycine is particularly preferred. The amino acid is included in the salt in an amount of 1-3 mol, preferably 1.3-1.8 mol, per mole of zirconium in each case.

Preferred antiperspirant salts are aluminum-zirconium tetrachlorohydrate (Al:Zr=2-6, M:Cl=0.9-1.3), in particular salts with a molar metal to chloride ratio of 0.9-1.1, preferably 0.9-1.0.

Preferred furthermore according to the invention are aluminum-zirconium chlorohydrate glycine salts which are stabilized with betaine ((CH₃)₃N⁺—CH₂—COO⁻). Particularly preferred corresponding compounds have an overall molar (betaine+glycine)/Zr ratio of (0.1-3.0):1, preferably (0.7-1.5):1, and a molar ratio of betaine to glycine of at least 0.001:1. In a particularly preferred embodiment of the invention, a so-called “activated” salt is included as a particularly effective antiperspirant salt, in particular one with a high HPLC peak 5 aluminum content, in particular with a peak 5 area of at least 33%, particularly preferably at least 45%, based on the total area under peaks 2-5, measured using HPLC in a 10% by weight of an aqueous solution of the active substance under conditions in which the aluminum species are resolved into at least 4 consecutive peaks (referred to as peaks 2-5).

Preferred further are activated “E⁵AZCH” salts with a high HPLC peak 4 to peak 3 area ratio of at least 0.4, preferably at least 0.7, particularly preferably at least 0.9.

Other particularly preferred active antiperspirant substances are the aluminum-zirconium salts with a high HPLC peak 5 aluminum content which are additionally stabilized with a water-soluble strontium salt and/or with a water-soluble calcium salt.

Particularly preferred compositions of the invention are characterized in that the at least one active antiperspirant substance is included in a total amount of 5-40% by weight, preferably 10-35% by weight, particularly preferably 15-28% by weight, and exceptionally preferably 23-27% by weight, based on the total weight of the active substance (USP), free from water of crystallization, in the composition, without taking into account optionally present propellants.

In a further particularly preferred embodiment, the compositions of the invention can include both at least one active deodorant substance and at least one active antiperspirant substance.

As other ingredients the compositions of the invention include a mixture of at least three oils, liquid under normal conditions, which represent a carrier fluid or a suspending medium with a reduced textile staining potential for the at least one active antiperspirant substance.

In the case of cosmetic oils, a distinction is made between volatile and non-volatile oils. Non-volatile oils are understood to mean oils that at 20° C. and an ambient pressure of 1013 hPa have a vapor pressure of less than 2.66 Pa (0.02 mm Hg). Volatile oils are understood to be those oils that at 20° C. and an ambient pressure of 1013 hPa have a vapor pressure of 2.66 Pa-40,000 Pa (0.02 mm-300 mm Hg), preferably 12-12,000 Pa (0.1-90 mm Hg), particularly preferably 13-8000 Pa, exceptionally preferably 30-3000 Pa, more preferably 100-400 Pa.

Compositions of the invention include at least one volatile silicone oil in a total amount of 28 to 61% by weight, preferably 30 to 50% by weight, particularly preferably 32 to 45% by weight, exceptionally preferably 35 to 38% by weight, wherein all quantities given in percentage by weight in each case refer to the weight of the composition, without taking into account optionally present propellants.

Preferred volatile silicone oils are selected from dialkyl- and alkylarylsiloxanes, which at 20° C. and an ambient pressure of 1013 hPa have a vapor pressure of less than 2.66 Pa (0.02 mm Hg), such as, for example, octamethylcyclotetrasiloxane, decamethylcyclopentasiloxane, dodecamethylcyclohexasiloxane, dimethylpolysiloxane, low-molecular-weight phenyl trimethicone, and methylphenyl polysiloxane, but hexamethyldisiloxane, octamethyltrisiloxane, and decamethyltetrasiloxane as well. Volatile silicone oils, which may be cyclic, are particularly preferred such as, e.g., octamethylcyclotetrasiloxane, decamethylcyclopentasiloxane, and dodecamethylcyclohexasiloxane, as well as mixtures thereof such as those included, e.g., in the commercial products DC 244, 245, 344, and 345 from Dow Corning (vapor pressure of about 13-15 Pa at 20° C.).

Likewise, particularly preferred are volatile linear silicone oils with 2-10 siloxane units, in particular hexamethyldisiloxane (L₂), octamethyltrisiloxane (L₃), decamethyltetrasiloxane (L₄), as well as any two- or three-component mixtures of L₂, L₃, and/or L₄, preferably mixtures such as those included, e.g., in the commercial products DC 2-1184, Dow Corning® 200 (0.65 cSt), and Dow Corning® 200 (1.5 cSt) from Dow Corning. Another preferred volatile silicone oil is a low-molecular-weight phenyl trimethicone with a vapor pressure of about 2000 Pa at 20° C., as can be obtained, e.g., from GE Bayer Silicones/Momentive under the name Baysilone Fluid PD 5.

Volatile silicone oils are excellently suitable carrier oils for antiperspirant compositions of the invention, because they provide them with a pleasant skin feel and low staining of textiles.

Antiperspirant compositions of the invention are therefore characterized in that the at least one volatile silicone oil is selected from octamethylcyclotetrasiloxane, decamethylcyclopentasiloxane, dodecamethylcyclohexasiloxane, hexamethyldisiloxane, octamethyltrisiloxane, and decamethyltetrasiloxane, and mixtures thereof, in particular decamethylcyclopentasiloxane, mixtures of decamethylcyclopentasiloxane and dodecamethylcyclohexasiloxane, and mixtures of hexamethyldisiloxane, octamethyltrisiloxane, and decamethyltetrasiloxane, particularly preferably selected from decamethylcyclopentasiloxane.

Other antiperspirant compositions of the invention are characterized by a total content of at least one volatile silicone oil of 28 to 61% by weight, preferably 30 to 50% by weight, particularly preferably 32 to 45% by weight, exceptionally preferably 35 to 38% by weight, wherein all quantities given in percentage by weight in each case refer to the weight of the antiperspirant composition, without taking into account optionally present propellants.

Antiperspirant compositions particularly preferred according to the invention are characterized in that they include as oil component c1) 28 to 61% by weight, preferably 30 to 50% by weight, particularly preferably 32 to 45% by weight, exceptionally preferably 35 to 38% by weight of cyclopentasiloxane, wherein all quantities given in percentage by weight in each case refer to the weight of the antiperspirant composition, without taking into account optionally present propellants.

Apart from the at least one volatile silicone oil, the compositions of the invention include 7 to 20% by weight, preferably 8 to 17% by weight, particularly preferably 11 to 16% by weight, exceptionally preferably 12 to 13% by weight of PPG-14 butyl ether, wherein all quantities given in percentage by weight in each case refer to the weight of the antiperspirant composition, without taking into account optionally present propellants.

In addition to the at least one volatile silicone oil and PPG-14 butyl ether, the compositions of the invention contain

0 to 18% by weight, preferably 0.1 to 15% by weight, particularly preferably 0.5 to 13% by weight, exceptionally preferably 1 to 7% by weight of 2-phenoxyethyl octanoate, and
0 to 7% by weight, preferably 0.3 to 6% by weight, particularly preferably 1 to 5% by weight, exceptionally preferably 2 to 4% by weight of PPG-3 myristyl ether,
with the proviso that at least one of the oils, 2-phenoxyethyl octanoate or PPG-3 myristyl ether, is included, and with the further proviso that the total amount of PPG-14 butyl ether and 2-phenoxyethyl octanoate and PPG-3 myristyl ether is a maximum of 30% by weight, wherein all quantities given in percentage by weight in each case refer to the weight of the composition, without taking into account optionally present propellants.

Surprisingly it was found that an especially satisfactory balance between low fiber or textile staining, primarily wash-resistant fiber or textile staining, on the one hand, and good masking of visible residues, on the other, are obtainable with the oil mixtures of the invention.

As a result of the relatively rapid evaporation of the at least one volatile silicone oil, solid, insoluble components, in particular the active antiperspirant substances, can be visible on the skin as an unattractive residue. These residues can be successfully masked with the oil combination c2 and [c3 and/or c4] of the invention. The at least one nonvolatile oil, which is different from c1, can support c1 in compensating for the negative effect of the volatile oil on the residue behavior of the antiperspirant compositions preferred according to the invention. In addition, parameters such as skin feel, visibility of the residue, and stability of the suspension can be finely regulated and better adapted to the user's requirements with a mixture of a nonvolatile and volatile oil.

Compositions very particularly preferred according to the invention contain, in each case based on the weight of the composition, without taking into account optionally present propellants:
7 to 20% by weight of PPG-14 butyl ether and 0.1 to 18% by weight of 2-phenoxyethyl octanoate and 28 to 61% by weight of cyclopentasiloxane or
7 to 20% by weight of PPG-14 butyl ether and 0.1 to 15% by weight of 2-phenoxyethyl octanoate and 28 to 61% by weight of cyclopentasiloxane or
7 to 20% by weight of PPG-14 butyl ether and 0.5 to 13% by weight of 2-phenoxyethyl octanoate and 28 to 61% by weight of cyclopentasiloxane or
7 to 20% by weight of PPG-14 butyl ether and 1 to 7% by weight of 2-phenoxyethyl octanoate and 28 to 61% by weight of cyclopentasiloxane or
8 to 17% by weight of PPG-14 butyl ether and 0.1 to 18% by weight of 2-phenoxyethyl octanoate and 28 to 61% by weight of cyclopentasiloxane or
8 to 17% by weight of PPG-14 butyl ether and 0.1 to 15% by weight of 2-phenoxyethyl octanoate and 28 to 61% by weight of cyclopentasiloxane or
8 to 17% by weight of PPG-14 butyl ether and 0.5 to 13% by weight of 2-phenoxyethyl octanoate and 28 to 61% by weight of cyclopentasiloxane or
8 to 17% by weight of PPG-14 butyl ether and 1 to 7% by weight of 2-phenoxyethyl octanoate and 28 to 61% by weight of cyclopentasiloxane or
7 to 20% by weight of PPG-14 butyl ether and 0.1 to 18% by weight of 2-phenoxyethyl octanoate and 30 to 50% by weight of cyclopentasiloxane or
7 to 20% by weight of PPG-14 butyl ether and 0.1 to 15% by weight of 2-phenoxyethyl octanoate and 30 to 50% by weight of cyclopentasiloxane or
7 to 20% by weight of PPG-14 butyl ether and 0.5 to 13% by weight of 2-phenoxyethyl octanoate and 30 to 50% by weight of cyclopentasiloxane or
7 to 20% by weight of PPG-14 butyl ether and 1 to 7% by weight of 2-phenoxyethyl octanoate and 30 to 50% by weight of cyclopentasiloxane or
8 to 17% by weight of PPG-14 butyl ether and 0.1 to 18% by weight of 2-phenoxyethyl octanoate and 32 to 45% by weight of cyclopentasiloxane or
8 to 17% by weight of PPG-14 butyl ether and 0.1 to 15% by weight of 2-phenoxyethyl octanoate and 32 to 45% by weight of cyclopentasiloxane or
8 to 17% by weight of PPG-14 butyl ether and 0.5 to 13% by weight of 2-phenoxyethyl octanoate and 32 to 45% by weight of cyclopentasiloxane or
8 to 17% by weight of PPG-14 butyl ether and 1 to 7% by weight of 2-phenoxyethyl octanoate and 32 to 45% by weight of cyclopentasiloxane or
8 to 17% by weight of PPG-14 butyl ether and 0.1 to 18% by weight of 2-phenoxyethyl octanoate and 30 to 35% by weight of cyclopentasiloxane or
8 to 17% by weight of PPG-14 butyl ether and 0.1 to 15% by weight of 2-phenoxyethyl octanoate and 30 to 35% by weight of cyclopentasiloxane or
8 to 17% by weight of PPG-14 butyl ether and 0.5 to 13% by weight of 2-phenoxyethyl octanoate and 30 to 35% by weight of cyclopentasiloxane or
8 to 17% by weight of PPG-14 butyl ether and 1 to 7% by weight of 2-phenoxyethyl octanoate and 30 to 35% by weight of cyclopentasiloxane or
7 to 20% by weight of PPG-14 butyl ether and 0.3 to 7% by weight of PPG-3 myristyl ether and 28 to 61% by weight of cyclopentasiloxane or
7 to 20% by weight of PPG-14 butyl ether and 0.3 to 6% by weight of PPG-3 myristyl ether and 28 to 61% by weight of cyclopentasiloxane or
7 to 20% by weight of PPG-14 butyl ether and 1 to 5% by weight of PPG-3 myristyl ether and 28 to 61% by weight of cyclopentasiloxane or
7 to 20% by weight of PPG-14 butyl ether and 2 to 4% by weight of PPG-3 myristyl ether and 28 to 61% by weight of cyclopentasiloxane or
8 to 17% by weight of PPG-14 butyl ether and 0.3 to 7% by weight of PPG-3 myristyl ether and 28 to 61% by weight of cyclopentasiloxane or
8 to 17% by weight of PPG-14 butyl ether and 0.3 to 6% by weight of PPG-3 myristyl ether and 28 to 61% by weight of cyclopentasiloxane or
8 to 17% by weight of PPG-14 butyl ether and 1 to 5% by weight of PPG-3 myristyl ether and 28 to 61% by weight of cyclopentasiloxane or
8 to 17% by weight of PPG-14 butyl ether and 2 to 4% by weight of PPG-3 myristyl ether and 28 to 61% by weight of cyclopentasiloxane or
7 to 20% by weight of PPG-14 butyl ether and 0.3 to 7% by weight of PPG-3 myristyl ether and 30 to 50% by weight of cyclopentasiloxane or
7 to 20% by weight of PPG-14 butyl ether and 0.3 to 6% by weight of PPG-3 myristyl ether and 30 to 50% by weight of cyclopentasiloxane or
7 to 20% by weight of PPG-14 butyl ether and 1 to 5% by weight of PPG-3 myristyl ether and 30 to 50% by weight of cyclopentasiloxane or
7 to 20% by weight of PPG-14 butyl ether and 2 to 4% by weight of PPG-3 myristyl ether and 30 to 50% by weight of cyclopentasiloxane or
8 to 17% by weight of PPG-14 butyl ether and 0.3 to 7% by weight of PPG-3 myristyl ether and 32 to 45% by weight of cyclopentasiloxane or
8 to 17% by weight of PPG-14 butyl ether and 0.3 to 6% by weight of PPG-3 myristyl ether and 32 to 45% by weight of cyclopentasiloxane or
8 to 17% by weight of PPG-14 butyl ether and 1 to 5% by weight of PPG-3 myristyl ether and 32 to 45% by weight of cyclopentasiloxane or
8 to 17% by weight of PPG-14 butyl ether and 2 to 4% by weight of PPG-3 myristyl ether and 32 to 45% by weight of cyclopentasiloxane or
8 to 17% by weight of PPG-14 butyl ether and 0.3 to 7% by weight of PPG-3 myristyl ether and 30 to 35% by weight of cyclopentasiloxane or
8 to 17% by weight of PPG-14 butyl ether and 0.3 to 6% by weight of PPG-3 myristyl ether and 30 to 35% by weight of cyclopentasiloxane or
8 to 17% by weight of PPG-14 butyl ether and 1 to 5% by weight of PPG-3 myristyl ether and 30 to 35% by weight of cyclopentasiloxane or
8 to 17% by weight of PPG-14 butyl ether and 2 to 4% by weight of PPG-3 myristyl ether and 30 to 35% by weight of cyclopentasiloxane or
7 to 20% by weight of PPG-14 butyl ether and 0.1 to 18% by weight of 2-phenoxyethyl octanoate and 0.3 to 7% by weight of PPG-3 myristyl ether and 28 to 61% by weight of cyclopentasiloxane or
7 to 20% by weight of PPG-14 butyl ether and 0.1 to 15% by weight of 2-phenoxyethyl octanoate and 0.3 to 7% by weight of PPG-3 myristyl ether and 28 to 61% by weight of cyclopentasiloxane or
7 to 20% by weight of PPG-14 butyl ether and 0.5 to 13% by weight of 2-phenoxyethyl octanoate and 0.3 to 7% by weight of PPG-3 myristyl ether and 28 to 61% by weight of cyclopentasiloxane or
7 to 20% by weight of PPG-14 butyl ether and 1 to 7% by weight of 2-phenoxyethyl octanoate and 0.3 to 7% by weight of PPG-3 myristyl ether and 28 to 61% by weight of cyclopentasiloxane or
8 to 17% by weight of PPG-14 butyl ether and 0.1 to 18% by weight of 2-phenoxyethyl octanoate and 0.3 to 6% by weight of PPG-3 myristyl ether and 28 to 61% by weight of cyclopentasiloxane or
8 to 17% by weight of PPG-14 butyl ether and 0.1 to 15% by weight of 2-phenoxyethyl octanoate and 0.3 to 6% by weight of PPG-3 myristyl ether and 28 to 61% by weight of cyclopentasiloxane or
8 to 17% by weight of PPG-14 butyl ether and 0.5 to 13% by weight of 2-phenoxyethyl octanoate and 0.3 to 6% by weight of PPG-3 myristyl ether and 28 to 61% by weight of cyclopentasiloxane or
8 to 17% by weight of PPG-14 butyl ether and 1 to 7% by weight of 2-phenoxyethyl octanoate and 0.3 to 6% by weight of PPG-3 myristyl ether and 28 to 61% by weight of cyclopentasiloxane or
7 to 20% by weight of PPG-14 butyl ether and 0.1 to 18% by weight of 2-phenoxyethyl octanoate and 1 to 5% by weight of PPG-3 myristyl ether and 30 to 50% by weight of cyclopentasiloxane or
7 to 20% by weight of PPG-14 butyl ether and 0.1 to 15% by weight of 2-phenoxyethyl octanoate and 1 to 5% by weight of PPG-3 myristyl ether and 30 to 50% by weight of cyclopentasiloxane or
7 to 20% by weight of PPG-14 butyl ether and 0.5 to 13% by weight of 2-phenoxyethyl octanoate and 1 to 5% by weight of PPG-3 myristyl ether and 30 to 50% by weight of cyclopentasiloxane or
7 to 20% by weight of PPG-14 butyl ether and 1 to 7% by weight of 2-phenoxyethyl octanoate and 1 to 5% by weight of PPG-3 myristyl ether and 30 to 50% by weight of cyclopentasiloxane or
8 to 17% by weight of PPG-14 butyl ether and 0.1 to 18% by weight of 2-phenoxyethyl octanoate and 2 to 4% by weight of PPG-3 myristyl ether and 32 to 45% by weight of cyclopentasiloxane or
8 to 17% by weight of PPG-14 butyl ether and 0.1 to 15% by weight of 2-phenoxyethyl octanoate and 2 to 4% by weight of PPG-3 myristyl ether and 32 to 45% by weight of cyclopentasiloxane or
8 to 17% by weight of PPG-14 butyl ether and 0.5 to 13% by weight of 2-phenoxyethyl octanoate and 2 to 4% by weight of PPG-3 myristyl ether and 32 to 45% by weight of cyclopentasiloxane or
8 to 17% by weight of PPG-14 butyl ether and 1 to 7% by weight of 2-phenoxyethyl octanoate and 2 to 4% by weight of PPG-3 myristyl ether and 32 to 45% by weight of cyclopentasiloxane or
8 to 17% by weight of PPG-14 butyl ether and 0.1 to 18% by weight of 2-phenoxyethyl octanoate and 2 to 4% by weight of PPG-3 myristyl ether and 30 to 35% by weight of cyclopentasiloxane or
8 to 17% by weight of PPG-14 butyl ether and 0.1 to 15% by weight of 2-phenoxyethyl octanoate and 2 to 4% by weight of PPG-3 myristyl ether and 30 to 35% by weight of cyclopentasiloxane or
8 to 17% by weight of PPG-14 butyl ether and 0.5 to 13% by weight of 2-phenoxyethyl octanoate and 2 to 4% by weight of PPG-3 myristyl ether and 30 to 35% by weight of cyclopentasiloxane or
8 to 17% by weight of PPG-14 butyl ether and 1 to 7% by weight of 2-phenoxyethyl octanoate and 2 to 4% by weight of PPG-3 myristyl ether and 30 to 35% by weight of cyclopentasiloxane.

In addition to the aforementioned oil mixtures, the compositions of the invention can include further at least one other oil, which is different from the oils c1, c2, c3, and c4 and is called an “additional oil” below, namely, preferably in a total amount of 0 to 12% by weight, particularly preferably 0.1 to 11% by weight, exceptionally preferably 3 to 10% by weight, based in each case on the weight of the composition, without taking into account optionally present propellants.

Additional oils particularly preferred according to the invention are selected from the esters of linear or branched, saturated or unsaturated fatty alcohols having 2 to 30 carbon atoms with linear or branched, saturated or unsaturated fatty acids having 2 to 30 carbon atoms, which may be hydroxylated. Among these, isopropyl myristate, isopropyl palmitate, isopropyl stearate, 2-ethylhexyl palmitate, and 2-ethylhexyl stearate are exceptionally preferred. Likewise preferred are 2-hexyldecyl stearate, 2-hexyldecyl laurate, isononyl isononanoate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-hexyl laurate, n-decyl oleate, oleyl oleate, oleyl erucate, erucyl oleate, and erucyl erucate, as well as mixtures of the aforementioned esters.

Other additional oils preferred according to the invention are selected from the mono- and polyesters of lactic acid, citric acid, tartaric acid, or adipic acid with a monohydric alcohol having 2 to 9 carbon atoms. A particularly preferred ester of this type is triethyl citrate.

Additional oils exceptionally preferred according to the invention are selected from isopropyl myristate, isopropyl palmitate, isopropyl stearate, 2-ethylhexyl palmitate, 2-ethylhexyl stearate, and triethyl citrate, as well as mixtures of the aforementioned esters.

Compositions exceptionally preferred according to the invention include at least one oil, selected from isopropyl myristate, isopropyl palmitate, isopropyl stearate, 2-ethylhexyl palmitate, 2-ethylhexyl stearate, and triethyl citrate, as well as mixtures of the aforementioned esters, in a total amount of 0 to 12% by weight, particularly preferably 0.1 to 11% by weight, exceptionally preferably 3 to 10% by weight, based in each case on the weight of the composition, without taking into account optionally present propellants.

Other additional oils preferred according to the invention are selected from dicarboxylic acid esters of linear or branched C₂-C₁₀ alkanols, particularly diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate, and di-(2-hexyldecyl) succinate, as well as mixtures of the aforesaid esters.

Other additional oils preferred according to the invention are selected from the benzoic acid esters of linear or branched C₈₋₂₂ alkanols. Preferred alkyl benzoates are dodecyl benzoate, tridecyl benzoate, tetradecyl benzoate, pentadecyl benzoate, hexadecyl benzoate, octadecyl benzoate, 2-methylheptadecyl benzoate, and octyldodecyl benzoate. Particularly preferred are benzoic acid-C₁₂-C₁₅ alkyl esters, e.g., obtainable as the commercial product Finsolv® TN, benzoic acid isostearyl ester, 2-ethylhexyl benzoate, and benzoic acid-2-octyldodecyl ester, benzoic acid-C₁₂-C₁₅ alkyl esters being exceptionally preferred.

Other additional oils preferred according to the invention are selected from mono- and polyesters of lactic acid, citric acid, tartaric acid, or adipic acid with a dihydric, trihydric, or tetrahydric alcohol having 2 to 9 carbon atoms. Particularly preferred esters of this type are selected from ethylene glycol monolactate, ethylene glycol monocitrate, ethylene glycol monotartrate, ethylene glycol monoadipate, ethylene glycol dilactate, ethylene glycol dicitrate, ethylene glycol ditartrate, ethylene glycol diadipate, 1,2-propylene glycol monolactate, 1,2-propylene glycol monocitrate, 1,2-propylene glycol monotartrate, 1,2-propylene glycol monoadipate, 1,2-propylene glycol dilactate, 1,2-propylene glycol dicitrate, 1,2-propylene glycol ditartrate, 1,2-propylene glycol diadipate, 1,3-propylene glycol monolactate, 1,3-propylene glycol monocitrate, 1,3-propylene glycol monotartrate, 1,3-propylene glycol monoadipate, 1,3-propylene glycol dilactate, 1,3-propylene glycol dicitrate, 1,3-propylene glycol ditartrate, 1,3-propylene glycol diadipate, 1,2-butylene glycol monolactate, 1,2-butylene glycol monocitrate, 1,2-butylene glycol monotartrate, 1,2-butylene glycol monoadipate, 1,2-butylene glycol dilactate, 1,2-butylene glycol dicitrate, 1,2-butylene glycol ditartrate, 1,2-butylene glycol diadipate, 1,3-butylene glycol monolactate, 1,3-butylene glycol monocitrate, 1,3-butylene glycol monotartrate, 1,3-butylene glycol monoadipate, 1,3-butylene glycol dilactate, 1,3-butylene glycol dicitrate, 1,3-butylene glycol ditartrate, 1,3-butylene glycol diadipate, 1,4-butylene glycol monolactate, 1,4-butylene glycol monocitrate, 1,4-butylene glycol monotartrate, 1,4-butylene glycol monoadipate, 1,4-butylene glycol dilactate, 1,4-butylene glycol dicitrate, 1,4-butylene glycol ditartrate, 1,4-butylene glycol diadipate, 1,2-pentanediol monolactate, 1,2-pentanediol monocitrate, 1,2-pentanediol monotartrate, 1,2-pentanediol monoadipate, 1,2-pentanediol dilactate, 1,2-pentanediol dicitrate, 1,2-pentanediol ditartrate, 1,2-pentanediol diadipate, 1,3-pentanediol monolactate, 1,3-pentanediol monocitrate, 1,3-pentanediol monotartrate, 1,3-pentanediol monoadipate, 1,3-pentanediol dilactate, 1,3-pentanediol dicitrate, 1,3-pentanediol ditartrate, 1,3-pentanediol diadipate, 1,4-pentanediol monolactate, 1,4-pentanediol monocitrate, 1,4-pentanediol monotartrate, 1,4-pentanediol monoadipate, 1,4-pentanediol dilactate, 1,4-pentanediol dicitrate, 1,4-pentanediol ditartrate, 1,4-pentanediol diadipate, 1,5-pentanediol monolactate, 1,5-pentanediol monocitrate, 1,5-pentanediol monotartrate, 1,5-pentanediol monoadipate, 1,5-pentanediol dilactate, 1,5-pentanediol dicitrate, 1,5-pentanediol ditartrate, 1,5-pentanediol diadipate, 1,2-hexanediol monolactate, 1,2-hexanediol monocitrate, 1,2-hexanediol monotartrate, 1,2-hexanediol monoadipate, 1,2-hexanediol dilactate, 1,2-hexanediol dicitrate, 1,2-hexanediol ditartrate, 1,2-hexanediol diadipate, 1,3-hexanediol monolactate, 1,3-hexanediol monocitrate, 1,3-hexanediol monotartrate, 1,3-hexanediol monoadipate, 1,3-hexanediol dilactate, 1,3-hexanediol dicitrate, 1,3-hexanediol ditartrate, 1,3-hexanediol diadipate, 1,4-hexanediol monolactate, 1,4-hexanediol monocitrate, 1,4-hexanediol monotartrate, 1,4-hexanediol monoadipate, 1,4-hexanediol dilactate, 1,4-hexanediol dicitrate, 1,4-hexanediol ditartrate, 1,4-hexanediol diadipate, 1,5-hexanediol monolactate, 1,5-hexanediol monocitrate, 1,5-hexanediol monotartrate, 1,5-hexanediol monoadipate, 1,5-hexanediol dilactate, 1,5-hexanediol dicitrate, 1,5-hexanediol ditartrate, 1,5-hexanediol diadipate, 1,6-hexanediol monolactate, 1,6-hexanediol monocitrate, 1,6-hexanediol monotartrate, 1,6-hexanediol monoadipate, 1,6-hexanediol dilactate, 1,6-hexanediol dicitrate, 1,6-hexanediol ditartrate, 1,6-hexanediol diadipate, 2-ethylhexane-1,2-diol monolactate, 2-ethylhexane-1,2-diol monocitrate, 2-ethylhexane-1,2-diol monotartrate, 2-ethylhexane-1,2-diol monoadipate, 2-ethylhexane-1,2-diol dilactate, 2-ethylhexane-1,2-diol dicitrate, 2-ethylhexane-1,2-diol ditartrate, 2-ethylhexane-1,2-diol diadipate, 2-ethylhexane-1,3-diol monolactate, 2-ethylhexane-1,3-diol monocitrate, 2-ethylhexane-1,3-diol monotartrate, 2-ethylhexane-1,3-diol monoadipate, 2-ethylhexane-1,3-diol dilactate, 2-ethylhexane-1,3-diol dicitrate, 2-ethylhexane-1,3-diol ditartrate, 2-ethylhexane-1,3-diol diadipate, 2-ethylhexane-1,4-diol monolactate, 2-ethylhexane-1,4-diol monocitrate, 2-ethylhexane-1,4-diol monotartrate, 2-ethylhexane-1,4-diol monoadipate, 2-ethylhexane-1,4-diol dilactate, 2-ethylhexane-1,4-diol dicitrate, 2-ethylhexane-1,4-diol ditartrate, 2-ethylhexane-1,4-diol diadipate, 2-ethylhexane-1,5-diol monolactate, 2-ethylhexane-1,5-diol monocitrate, 2-ethylhexane-1,5-diol monotartrate, 2-ethylhexane-1,5-diol monoadipate, 2-ethylhexane-1,5-diol dilactate, 2-ethylhexane-1,5-diol dicitrate, 2-ethylhexane-1,5-diol ditartrate, 2-ethylhexane-1,5-diol diadipate, 2-ethylhexane-1, 6-diol monolactate, 2-ethylhexane-1,6-diol monocitrate, 2-ethylhexane-1,6-diol monotartrate, 2-ethylhexane-1,6-diol monoadipate, 2-ethylhexane-1,6-diol dilactate, 2-ethylhexane-1,6-diol dicitrate, 2-ethylhexane-1,6-diol ditartrate, 2-ethylhexane-1,6-diol diadipate, 1,2-heptanediol monolactate, 1,2-heptanediol monocitrate, 1,2-heptanediol monotartrate, 1,2-heptanediol monoadipate, 1,2-heptanediol dilactate, 1,2-heptanediol dicitrate, 1,2-heptanediol ditartrate, 1,2-heptanediol diadipate, 1,3-heptanediol monolactate, 1,3-heptanediol monocitrate, 1,3-heptanediol monotartrate, 1,3-heptanediol monoadipate, 1,3-heptanediol dilactate, 1,3-heptanediol dicitrate, 1,3-heptanediol ditartrate, 1,3-heptanediol diadipate, 1,4-heptanediol monolactate, 1,4-heptanediol monocitrate, 1,4-heptanediol monotartrate, 1,4-heptanediol monoadipate, 1,4-heptanediol dilactate, 1,4-heptanediol dicitrate, 1,4-heptanediol ditartrate, 1,4-heptanediol diadipate, 1,5-heptanediol monolactate, 1,5-heptanediol monocitrate, 1,5-heptanediol monotartrate, 1,5-heptanediol monoadipate, 1,5-heptanediol dilactate, 1,5-heptanediol dicitrate, 1,5-heptanediol ditartrate, 1,5-heptanediol diadipate, 1,6-heptanediol monolactate, 1,6-heptanediol monocitrate, 1,6-heptanediol monotartrate, 1,6-heptanediol monoadipate, 1,6-heptanediol dilactate, 1,6-heptanediol dicitrate, 1,6-heptanediol ditartrate, 1,6-heptanediol diadipate, 1,7-heptanediol monolactate, 1,7-heptanediol monocitrate, 1,7-heptanediol monotartrate, 1,7-heptanediol monoadipate, 1,7-heptanediol dilactate, 1,7-heptanediol dicitrate, 1,7-heptanediol ditartrate, 1,7-heptanediol diadipate, 1,2-octanediol monolactate, 1,2-octanediol monocitrate, 1,2-octanediol monotartrate, 1,2-octanediol monoadipate, 1,2-octanediol dilactate, 1,2-octanediol dicitrate, 1,2-octanediol ditartrate, 1,2-octanediol diadipate, 1,3-octanediol monolactate, 1,3-octanediol monocitrate, 1,3-octanediol monotartrate, 1,3-octanediol monoadipate, 1,3-octanediol dilactate, 1,3-octanediol dicitrate, 1,3-octanediol ditartrate, 1,3-octanediol diadipate, 1,4-octanediol monolactate, 1,4-octanediol monocitrate, 1,4-octanediol monotartrate, 1,4-octanediol monoadipate, 1,4-octanediol dilactate, 1,4-octanediol dicitrate, 1,4-octanediol ditartrate, 1,4-octanediol diadipate, 1,5-octanediol monolactate, 1,5-octanediol monocitrate, 1,5-octanediol monotartrate, 1,5-octanediol monoadipate, 1,5-octanediol dilactate, 1,5-octanediol dicitrate, 1,5-octanediol ditartrate, 1,5-octanediol diadipate, 1,6-octanediol monolactate, 1,6-octanediol monocitrate, 1,6-octanediol monotartrate, 1,6-octanediol monoadipate, 1,6-octanediol dilactate, 1,6-octanediol dicitrate, 1,6-octanediol ditartrate, 1,6-octanediol diadipate, 1,7-octanediol monolactate, 1,7-octanediol monocitrate, 1,7-octanediol monotartrate, 1,7-octanediol monoadipate, 1,7-octanediol dilactate, 1,7-octanediol dicitrate, 1,7-octanediol ditartrate, 1,7-octanediol diadipate, 1, 8-octanediol monolactate, 1,8-octanediol monocitrate, 1,8-octanediol monotartrate, 1,8-octanediol monoadipate, 1,8-octanediol dilactate, 1,8-octanediol dicitrate, 1,8-octanediol ditartrate, 1,8-octanediol diadipate, 2-methyl-1,3-propanediol monolactate, 2-methyl-1,3-propanediol monocitrate, 2-methyl-1,3-propanediol monotartrate, 2-methyl-1,3-propanediol monoadipate, 2-methyl-1,3-propanediol dilactate, 2-methyl-1,3-propanediol dicitrate, 2-methyl-1,3-propanediol ditartrate, 2-methyl-1,3-propanediol diadipate, dipropylene glycol monolactate, dipropylene glycol monotartrate, dipropylene glycol monocitrate, dipropylene glycol monoadipate, dipropylene glycol dilactate, dipropylene glycol ditartrate, dipropylene glycol dicitrate, dipropylene glycol diadipate, glycerol monolactate, glycerol monotartrate, glycerol monocitrate, glycerol monoadipate, glycerol dilactate, glycerol ditartrate, glycerol dicitrate, glycerol diadipate, glycerol trilactate, glycerol tritartrate, glycerol tricitrate, glycerol triadipate, diglycerol monolactate, diglycerol monotartrate, diglycerol monocitrate, diglycerol monoadipate, diglycerol dilactate, diglycerol ditartrate, diglycerol dicitrate, diglycerol diadipate, diglycerol trilactate, diglycerol tritartrate, diglycerol tricitrate, diglycerol triadipate, tripropylene glycol monolactate, tripropylene glycol monotartrate, tripropylene glycol monocitrate, tripropylene glycol monoadipate, tripropylene glycol dilactate, tripropylene glycol ditartrate, tripropylene glycol dicitrate, tripropylene glycol diadipate, tripropylene glycol trilactate, tripropylene glycol tritartrate, tripropylene glycol tricitrate, tripropylene glycol triadipate, triglycerol monolactate, triglycerol monotartrate, triglycerol monocitrate, triglycerol monoadipate, triglycerol dilactate, triglycerol ditartrate, triglycerol dicitrate, triglycerol diadipate, triglycerol trilactate, triglycerol tritartrate, triglycerol tricitrate, triglycerol triadipate, 1,2,6-hexanetriol monolactate, 1,2,6-hexanetriol monotartrate, 1,2,6-hexanetriol monocitrate, 1,2,6-hexanetriol monoadipate, 1,2,6-hexanetriol dilactate, 1,2,6-hexanetriol ditartrate, 1,2,6-hexanetriol dicitrate, 1,2,6-hexanetriol diadipate, 1,2,6-hexanetriol trilactate, 1,2,6-hexanetriol tritartrate, 1,2,6-hexanetriol tricitrate, 1,2,6-hexanetriol triadipate, trimethylolpropane monolactate, trimethylolpropane monotartrate, trimethylolpropane monocitrate, trimethylolpropane monoadipate, trimethylolpropane dilactate, trimethylolpropane ditartrate, trimethylolpropane dicitrate, trimethylolpropane diadipate, trimethylolpropane trilactate, trimethylolpropane tritartrate, trimethylolpropane tricitrate, trimethylolpropane triadipate, trimethylolethane monolactate, trimethylolethane monotartrate, trimethylolethane monocitrate, trimethylolethane monoadipate, trimethylolethane dilactate, trimethylolethane ditartrate, trimethylolethane dicitrate, trimethylolethane diadipate, trimethylolethane trilactate, trimethylolethane tritartrate, trimethylolethane tricitrate, and trimethylolethane triadipate, as well as mixtures thereof.

Triglyceride oils of linear or branched, saturated or unsaturated, optionally hydroxylated C₈₋₃₀ fatty acids, in particular natural oils, e.g., soybean oil, cottonseed oil, sunflower oil, palm oil, palm kernel oil, linseed oil, almond oil, castor oil, corn oil, rapeseed oil, olive oil, sesame oil, thistle oil, wheat germ oil, peach stone oil and the liquid fractions of coconut oil and the like, but also synthetic triglyceride oils, in particular capric/caprylic triglycerides, e.g., the commercial products Myritol® 318 or Myritol® 331 (BASF) with unbranched fatty acid esters and glyceryl triisostearin with branched fatty acid residues, are also suitable in principle as additional oils, but less preferred because of their more disadvantageous residue behavior. Triglyceride oils of this type should be included preferably only in a total amount of 0 to 1% by weight, particularly preferably 0 to 0.5% by weight, based in each case on the weight of the composition, without taking into account optionally present propellants.

Other additional oils preferred according to the invention are selected from symmetrical, asymmetrical, or cyclic esters of carbonic acid with fatty alcohols, e.g., dicaprylyl carbonate (Cetiol® CC), di-n-octyl carbonate, di-n-dodecyl carbonate, di(2-ethylhexyl)carbonate, or the esters according to the teaching of DE 19756454 A.

In addition to the aforementioned obligatory oil mixtures, at least one volatile non-silicone oil can also be included as an additional oil. Preferred volatile non-silicone oils are selected from C₈-C₁₆ isoparaffins, in particular from isodecane, isoundecane, isododecane, isotridecane, isotetradecane, isopentadecane, and isohexadecane, and mixtures thereof. C₁₀-C₁₃ isoparaffin mixtures are preferred, in particular those with a vapor pressure at 20° C. of about 10-400 Pa, preferably 13-300 Pa. This at least one volatile non-silicone oil, which is preferably selected from C₈-C₁₆ isoparaffins, in particular from isodecane, isoundecane, isododecane, isotridecane, isotetradecane, isopentadecane, and isohexadecane, as well as mixtures thereof, is preferably included in a total amount of 0.1 to 7% by weight, preferably 0.2 to 5% by weight, particularly preferably 0.5 to 2% by weight, based in each case on the total composition, optionally present propellants not being taken into account.

In addition to the aforementioned obligatory oil mixtures, at least one nonvolatile silicone oil can also be included as an additional oil. Preferred nonvolatile silicone oils are selected from linear polydimethylsiloxanes with kinematic viscosities (25° C.) in the range of 5-350 cSt, preferably 5-100 cSt, or also 10-50 cSt.

In addition to the aforementioned obligatory oil mixtures, at least one natural or synthetic hydrocarbon oil, selected from paraffin oils, C₁₈-C₃₀ isoparaffins, in particular isoeicosane, polyisobutene or polydecene, which are available, for example, under the name Emery® 3004, 3006, 3010 or under the name Ethylflo® from Albemarle or Nexbase® 2004G from Nestle, as well as 1,3-di(2-ethylhexyl)cyclohexane (available, e.g., under the trade name Cetiol® S from BASF), can also be included, preferably in a total amount of 0.1 to 7% by weight, preferably 0.2 to 5% by weight, particularly preferably 0.5 to 2% by weight, based in each case on the total composition, optionally present propellants not being taken into account.

Other additional oils preferred according to the invention are selected from branched, saturated or unsaturated fatty alcohols having 6-30 carbon atoms. These alcohols are often also referred to as “Guerbet alcohols,” since they are obtainable by the Guerbet reaction. Preferred alcohol oils are 2-hexyldecanol, octyldodecanol (Eutanol® G), and 2-ethylhexyl alcohol.

Fragrances and odorants are not included as oils c) according to the invention.

The definition of an odorant in the context of the present invention corresponds to the definition conventional to one skilled in the art, as can be gathered from RÖMPP Chemie Lexikon [RÖMPP's Chemistry Lexicon], as of December 2007. Accordingly, an odorant is a chemical compound with an odor and/or taste, which excites the receptors of hair cells of the olfactory system (adequate stimulus). The physical and chemical properties required for this are a low molar mass of a maximum of 300 g/mol, a high vapor pressure, minimal water solubility and high lipid solubility, and a weak polarity and the presence of at least one osmophoric group in the molecule. To differentiate volatile, low-molecular-weight substances which are not regarded and used as an odorant, either conventionally and also in the context of the present invention, but primarily as a solvent, such as, e.g., ethanol, propanol, isopropanol, and acetone, from odorants according to the invention, odorants according to the invention have a molar mass of 74 to 300 g/mol, include at least one osmophoric group in the molecule, and have an odor and/or taste; in other words, they stimulate the receptors of the hair cells of the olfactory system. Examples of fragrance and odorant compounds of the ester type are benzyl acetate, phenoxyethyl isobutyrate, p-tert-butylcyclohexyl acetate, linalyl acetate, dimethyl benzyl carbinyl acetate (DMBCA), phenyl ethyl acetate, benzyl acetate, ethyl methyl phenyl glycinate, allyl cyclohexyl propionate, styrallyl propionate, benzyl salicylate, cyclohexyl salicylate, Floramat, Melusat, and Jasmecyclat. Examples of fragrance and odorant compounds of the ether type are benzyl ethyl ether and Ambroxan; examples of fragrance and odorant compounds of the aldehyde type are the linear alkanals having 8-18 C atoms, citral, citronellal, citronellyl oxyacetaldehyde, cyclamen aldehyde, lilial, and bourgeonal; examples of fragrance and odorant compounds of the ketone type are ionones, alpha-isomethyl ionone, and methyl cedryl ketone; examples of fragrance and odorant compounds of the alcohol type are anethol, citronellol, eugenol, geraniol, linalool, phenyl ethyl alcohol, and terpineol; examples of fragrance and odorant compounds of the terpene type are limonene and pinene. Examples of fragrance and odorant compounds are pine oil, citrus oil, jasmine oil, patchouli oil, rose oil, ylang-ylang oil, clary sage oil, chamomile oil, clove oil, mint oil, cinnamon leaf oil, lime blossom oil, juniper berry oil, vetiver oil, olibanum oil, galbanum oil, labdanum oil, orange blossom oil, neroli oil, orange-peel oil, and sandalwood oil, further essential oils such as angelica root oil, anise oil, arnica blossom oil, basil oil, bay oil, bergamot oil, champaca flower oil, silver fir oil, silver fir cone oil, elemi oil, eucalyptus oil, fennel oil, fir needle oil, geranium oil, gingergrass oil, guaiacum oil, gurjun balsam oil, helichrysum oil, ho oil, ginger oil, iris oil, cajeput oil, calamus oil, chamomile oil, camphor oil, canaga oil, cardamom oil, cassia oil, pine-needle oil, copaiva balsam oil, coriander oil, curled mint oil, caraway oil, cumin oil, lavender oil, lemongrass oil, lime oil, mandarin oil, melissa oil, musk seed oil, myrrh oil, clove oil, niaouli oil, orange oil, oregano oil, palmarosa oil, patchouli oil, Peru balsam oil, petitgrain oil, pepper oil, peppermint oil, pimento oil, pine oil, rose oil, rosemary oil, sandalwood oil, celery oil, spike oil, star anise oil, turpentine oil, thuja oil, thyme oil, verbena oil, juniper berry oil, wormwood oil, wintergreen oil, hyssop oil, cinnamon oil, citronella oil, lemon oil, and cypress oil. Other fragrance and odorant compounds are ambrettolide, α-amyl cinnamaldehyde, anethol, anisaldehyde, anisyl alcohol, anisole, anthranilic acid methyl ester, acetophenone, benzyl acetone, benzaldehyde, benzoic acid ethyl ester, benzophenone, benzyl alcohol, benzyl acetate, benzyl benzoate, benzyl formate, benzyl valeriate, borneol, bornyl acetate, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl n-amyl ketone, methyl anthranilic acid methyl ester, p-methyl acetophenone, methyl chavicol, p-methyl quinoline, methyl 3-naphthyl ketone, methyl n-nonyl acetaldehyde, methyl n-nonyl ketone, muscone, β-naphthol ethyl ether, β-naphthol methyl ether, nerol, nitrobenzene, n-nonyl aldehyde, nonyl alcohol, n-octyl aldehyde, p-oxyacetophenone, pentadecanolide, β-phenyl ethyl alcohol, phenyl acetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thymene, thymol, γ-undecalactone, vanillin, veratrum aldehyde, cinnamaldehyde, cinnamyl alcohol, cinnamic acid, cinnamic acid ethyl ester, and cinnamic acid benzyl ester.

Other (more volatile) odorants are alkyl isothiocyanates (alkyl mustard oils), butanedione, limonene, linalool, linalyl acetate and linalyl propionate, menthol, menthone, methyl-n-heptenone, phellandrene, phenylacetaldehyde, terpinyl acetate, citral, and citronellal.

Preferably, mixtures of different fragrances are used, which together produce an attractive scent note.

Suitable perfume oils can also include natural odorant mixtures, as are obtainable from plant or animal sources, e.g., pine, citrus, jasmine, rose, lily, or ylang oil. Essential oils with a low volatility, which are mainly used as aroma components, are also suitable as perfume oils, e.g., sage oil, chamomile oil, melissa oil, mint oil, cinnamon leaf oil, lime blossom oil, juniper berry oil, vetiver oil, olibanum oil, galbanum oil, laudanum oil, clove oil, isoeugenol, thyme oil, bergamot oil, geranium oil, and rose oil.

Preferred compositions of the invention are characterized in that at least one fragrance is included in a total amount of 0.1-15% by weight, preferably 0.5-10% by weight, particularly preferably 1-8% by weight, exceptionally preferably 2-7% by weight, further exceptionally preferably 3-6% by weight, based in each case on the total weight of the propellant-free composition.

Other compositions preferred according to the invention are characterized by a content of at least one so-called “skin-cooling active substance.” Skin-cooling active substances in the context of the present invention are understood to be active substances that, when applied to the skin, as a result of the surface anesthetizing and stimulation of cold-sensitive nerves in a migraine and the like, produce a pleasant cool sensation, even if the areas of skin being treated actually display a normal or elevated temperature. Compounds that, like 1-menthol, stimulate the heat receptors in the skin and mucous membranes such that a cool sensory impression results are regarded according to the invention as skin-cooling active substances. In particular the CMR-1 receptor (“cold- and menthol-sensitive receptor”), which belongs to the family of TRP channels, is stimulated by the active cooling substances, which produces an impression of cold.

Preferred skin-cooling active substances are menthol, isopulegol, and menthol derivatives, e.g., menthyl lactate, menthyl pyrrolidone carboxylic acid, menthyl methyl ether, menthoxypropanediol, menthone glycerol acetal (9-methyl-6-(1-methylethyl)-1,4-dioxaspiro(4.5)decane-2-methanol), monomenthyl succinate, and 2-hydroxymethyl-3,5,5-trimethylcyclohexanol. Particularly preferred skin-cooling active substances are menthol, isopulegol, menthyl lactate, menthoxypropanediol, and menthyl pyrrolidone carboxylic acid.

Preferred compositions of the invention include at least one skin-cooling active substance in a total amount of 0.01-1% by weight, preferably 0.02-0.5% by weight and particularly preferably 0.05-0.2% by weight, based in each case on the total weight of the (propellant-free) composition.

Preferred compositions of the invention are characterized in that at least one encapsulated active substance is included. The active substances that can advantageously be encapsulated are, in particular, fragrances, perfume oils, and/or skin-cooling active substances, but also other active skincare substances, such as vitamins, antioxidants, etc.

Water-soluble polymers, such as starch, physically and/or chemically modified starches, cellulose derivatives such as, e.g., carboxymethyl cellulose, methyl cellulose, hydroxyethyl cellulose, or hydroxypropyl methyl cellulose, carrageens, alginates, maltodextrins, dextrins, plant gums, pectins, xanthans, polyvinyl acetate and polyvinyl alcohol, polyvinylpyrrolidine, polyamides, polyesters, and homo- and copolymers of monomers selected from acrylic acid, methacrylic acid, maleic acid, fumaric acid, itaconic acid, and the esters and salts of these acids, and any mixtures of these polymers, are preferred as the capsule material.

Preferred capsule materials are chemically modified starches, in particular aluminum starch octenylsuccinate, e.g., the commercial product Dry Flo Plus from National Starch, or sodium starch octenylsuccinate, e.g., the commercial product Capsul from National Starch, furthermore, carboxymethyl cellulose, carboxymethyl cellulose, methyl cellulose, hydroxyethyl cellulose, and hydroxypropyl methyl cellulose, ethyl cellulose, e.g., the commercial product Tylose H 10 from Clariant, further carrageens, alginates, and maltodextrins, and any mixtures of these polymers.

In a further embodiment preferred according to the invention, the compositions of the invention include 0 to a maximum of 5% by weight of ethanol.

Furthermore, the compositions of the invention can include additional active deodorant substances. As active deodorant substances, it is possible to use antimicrobial, antibacterial, or bacteriostatic substances, antioxidants, or odor adsorbants (e.g., zinc ricinoleate). Suitable antimicrobial, antibacterial, or bacteriostatic substances are, in particular, organohalogen compounds and organohalides, quaternary ammonium compounds, a series of plant extracts, and zinc compounds. Halogenated phenol derivatives are preferred such as, e.g., hexachlorophene or Irgasan DP 300 (triclosan, 2,4,4′-trichloro-2′-hydroxydiphenyl ether), 3,4,4′-trichlorocarbonilide, chlorhexidine (1,1′-hexamethylene-bis[5-(4-chlorophenyl)]biguanide), chlorhexidine gluconate, benzalkonium halides, and cetylpyridinium chloride. In addition, sodium bicarbonate, sodium phenolsulfonate, and zinc phenolsulfonate and, e.g., the components of lime blossom oil can be used. More weakly effective antimicrobial substances, but which have a specific action against the gram-positive microbes responsible for the decomposition of sweat, can also be used as active deodorant substances. Benzyl alcohol can also be used as an active deodorant substance. Other antibacterially effective deodorants are lantibiotics, glycoglycerolipids, sphingolipids (ceramides), sterols, and other active substances that inhibit bacterial adhesion to the skin, e.g., glycosidases, lipases, proteases, carbohydrates, di- and oligosaccharide fatty acid esters, and alkylated mono- and oligosaccharides. Preferred active deodorant substances are long-chain diols, e.g., 1,2-alkane(C₅-C₁₈)diols, glycerol mono(C₈-C₁₈) fatty acid esters or, particularly preferably, glycerol mono(C₆-C₁₆)alkyl ethers, in particular 2-ethylhexyl glycerol ether, which are highly compatible with the skin and mucosa and are effective against corynebacteria, and further phenoxyethanol, phenoxyisopropanol (3-phenoxypropan-2-ol), anisyl alcohol, 2-methyl-5-phenylpentan-1-ol, 1,1-dimethyl-3-phenylpropan-1-ol, benzyl alcohol, 2-phenylethan-1-ol, 3-phenylpropan-1-ol, 4-phenylbutan-1-ol, 5-phenylpentan-1-ol, 2-benzylheptan-1-ol, 2,2-dimethyl-3-phenylpropan-1-ol, 2,2-dimethyl-3-(3′-methylphenyl)propan-1-ol, 2-ethyl-3-phenylpropan-1-ol, 2-ethyl-3-(3′-methylphenyl)propan-1-ol, 3-(3′-chlorophenyl)-2-ethylpropan-1-ol, 3-(2′-chlorophenyl)-2-ethylpropan-1-ol, 3-(4′-chlorophenyl)-2-ethylpropan-1-ol, 3-(3′,4′-dichlorophenyl)-2-ethylpropan-1-ol, 2-ethyl-3-(2′-methylphenyl)propan-1-ol, 2-ethyl-3-(4′-methylphenyl)propan-1-ol, 3-(3′,4′-dimethylphenyl)-2-ethylpropan-1-ol, 2-ethyl-3-(4′-methoxyphenyl)propan-1-ol, 3-(3′,4′-dimethoxyphenyl)-2-ethylpropan-1-ol, 2-allyl-3-phenylpropan-1-ol, and 2-n-pentyl-3-phenylpropan-1-ol.

Complex-forming substances can also support the deodorizing action by stably complexing the heavy metal ions that have an oxidative catalytic action (e.g., iron or copper). Suitable complexing agents are, e.g., the salts of ethylenediaminetetraacetic acid or of nitrilotriacetic acid and the salts of 1-hydroxyethane-1,1-diphosphonic acid.

The compositions of the invention, which are applied as a spray, are preferably formulated in accordance with the requirements of the desired spray application.

The compositions of the invention are present as a suspension; in other words, the active antiperspirant substance and optionally other insoluble components are suspended in a liquid or solid carrier. Liquid-disperse systems of this type, e.g., as a roll-on or as dispersions to be applied as a spray, should be shaken before application.

Preferred compositions of the invention can be packaged, e.g., in pump or squeeze dispensers, in particular in multi-chamber pump or squeeze dispensers. Such dispensers use air, in particular ambient air, as a propellant or deliver the composition of the invention by pumps.

In a further preferred embodiment of the invention, the composition is applied by means of a compressed or liquefied propellant. For this purpose, the composition of the invention is packaged together with a propellant in a spray can. The propellant and composition of the invention in this case can be present as a mixture. It is also possible, however, that the propellant and the composition of the invention are present separated from one another, e.g., in so-called bag-in-can spray cans.

All quantitative data, unless otherwise specified, relate to the weight of the propellant-free composition.

Packaging in a multi-chamber dispenser offers particular technical advantages.

The multi-chamber dispenser can also be used such that one chamber is filled with the composition of the invention, whereas another chamber includes the compressed propellant. One such multi-chamber dispenser is, for example, a “bag-in can” package.

The two chambers, however, may also be connected to one another such that the composition of the invention is divided into two sub-compositions which may be discharged simultaneously from the package, for example, from separate orifices or from a single orifice.

Other preferred compositions of the invention are characterized in that they are packaged with at least one propellant in a suitable pressure container.

Propellants (propellant gases) preferred according to the invention are selected from propane, propene, n-butane, isobutane, isobutene, n-pentane, pentene, isopentane, isopentane, methane, ethane, dimethyl ether, nitrogen, air, oxygen, laughing gas, dichlorofluoromethane, chlorodifluoromethane, chlorofluoromethane, 1,1,2,2-tetrachloro-1-fluoroethane, 1,1,1,2-tetrachloro-2-fluoroethane, 1,2,2-trichloro-1,1-difluoroethane, 1,1,2-trichloro-1,2-difluoroethane, 1,1,1-trichloro-2,2-difluoroethane, 2,2-dichloro-1,1,1-trifluoroethane, 1,2-dichloro-1,1,2-trifluoroethane, 2-chloro-1,1,1,2-tetrafluoroethane, 1-chloro-1,1,2,2-tetrafluoroethane, 1,1,2-trichloro-2-fluoroethane, 1,2-dichloro-1,2-difluoroethane, 1,2-dichloro-1,1-difluoroethane, 1-chloro-1,2,2-trifluoroethane, 2-chloro-1,1,1-trifluoroethane, 1-chloro-1,1,2-trifluoroethane, 1,2-dichloro-1-fluoroethane, 1,1-dichloro-1-fluoroethane, 2-chloro-1,1-difluoroethane, 1-chloro-1,1-difluoroethane, 1-chloro-2-fluoroethane, 1-chloro-1-fluoroethane, 2-chloro-1,1-difluoroethane, 1,1,1,3-tetrafluoroethane, heptafluoro-n-propane, perfluoroethane, monochlorodifluoromethane, and 1,1-difluoroethane, both individually and in combination.

Particularly preferred are propane, n-butane, isobutane, and, particularly preferably, mixtures of said propellant gases. Preferred, furthermore, are also 1,1-difluoroethane, propane, n-butane, isobutane, and mixtures of these propellants, in particular mixtures of 1,1-difluoroethane and n-butane.

Hydrophilic propellant gases such as, e.g., carbon dioxide, can also be used advantageously in the context of the present invention, if the proportion of hydrophilic gases is selected as low and a lipophilic propellant gas (e.g., propane/butane) is present in excess. Propane, n-butane, isobutane, and mixtures of said propellant gases are particularly preferred. It has emerged that the use of n-butane as the sole propellant gas can be particularly preferred according to the invention.

The amount of propellants is preferably 10-95% by weight, particularly preferably 30-90% by weight, and exceptionally preferably 60-86% by weight, and furthermore exceptionally preferably 70, 72, 74, 76, 78, 82, 84, or 85% by weight, based in each case on the total weight of the preparation consisting of the composition (suspension) of the invention and the propellant.

Vessels made of metal (aluminum, tinplate, tin), protected or non-splintering plastic, or glass that is externally coated with plastic may be used as compressed gas containers; compressive strength and breaking strength, corrosion resistance, ease of filling, as well as aesthetic aspects, handling, printability, etc., play a role in their selection. Special protective interior coatings assure corrosion resistance to the suspension of the invention. A protective interior coating preferred according to the invention is an epoxy-phenolic coating, as can be obtained under the name Hoba 7407 P. The valves that are used particularly preferably have an internally coated valve disc, the coating and valve material being mutually compatible. If aluminum valves are used, thus the valve discs thereof can be coated on the inside, e.g., with a Micoflex coating. If tinplate valves are used according to the invention, thus the valve discs thereof can be coated on the inside, e.g., with PET (polyethylene terephthalate).

The containers are equipped with a suitable spray head. Depending on the spray head, discharge rates, based on fully filled containers, of 0.1 g/s to 2.0 g/s are possible.

Compositions of the invention, which can be sprayed as a spray, preferably include at least one thickener, which is selected from hydrophobized clay minerals. Preferred hydrophobized clay minerals are selected from hydrophobized montmorillonites, hydrophobized hectorites, and hydrophobized bentonites, particularly preferably from disteardimonium hectorite, stearalkonium hectorite, quaternium-18 hectorite, and quaternium-18 bentonite. Compositions preferred according to the invention are characterized in that they include at least one hydrophobized clay mineral in a total amount of 0.5-10% by weight, preferably 1-7% by weight, particularly preferably 2-6% by weight, exceptionally preferably 2.5-4% by weight, based in each case on the total weight of the propellant-free composition of the invention. Other lipophilic thickener preferred according to the invention are selected from pyrogenic silicic acids, e.g., the commercial products of the Aerosil® series from Evonik. Particularly preferred are hydrophobized pyrogenic silicic acid, particularly preferably silica silylate and silica dimethyl silylate. Compositions preferred according to the invention are characterized in that they include at least one pyrogenic silicic acid, preferably at least one hydrophobized pyrogenic silicic acid, in a total amount of 0.5-10% by weight, preferably 0.8-5% by weight, particularly preferably 1-4% by weight, exceptionally preferably 1.5-2% by weight, based in each case on the total weight of the propellant-free composition of the invention. Other compositions preferred according to the invention are characterized in that they include at least one hydrophobized pyrogenic silicic acid and at least one hydrophilic silicic acid.

In another preferred embodiment of the invention, the compositions of the invention include 10-25% by weight of a fat component that is solid under normal conditions and is preferably selected from fat components with a melting point of at least 50° C. Preferably, the solid fat component is selected from stearyl alcohol, cetyl alcohol, arachidyl alcohol, and/or behenyl alcohol. Furthermore, a proportion of 0.5-10% by weight of at least one fat component with a melting point of about 55-120° C. can be selected. These include in particular waxes, such as castor wax, beeswax, and wax esters. Moreover, 0.5-8% by weight of at least one fat component with a melting point of about 25-35° C. can be included. Furthermore, 0.5-30% by weight of at least one filler can be included, which is typically selected from talc, cellulose powders, starches, and starch derivatives. Furthermore, 0.1-10% by weight, preferably 1-5% by weight, particularly preferably 2-4% by weight of at least one oil-in-water emulsifier can be included.

Preferred compositions of the invention, which include 10-25% by weight of a fat component that is solid under normal conditions, are present in stick form.

A further subject of the present invention is a non-therapeutic cosmetic method for reducing and/or regulating sweat formation and/or body odor, in which method a composition of the invention or preferred according to the invention according to one of claims 1 to 9 is applied in an effective amount to skin, preferably to the skin in the axillary area.

The statements made regarding the compositions of the invention apply mutatis mutandis with respect to other preferred embodiments of the method of the invention.

A further subject of the present invention is the use of a mixture of
c1) at least one volatile silicone oil in a total amount of 28 to 61% by weight, preferably 30 to 50% by weight, particularly preferably 32 to 45% by weight, exceptionally preferably 35 to 38% by weight, furthermore
c2) 7 to 20% by weight, preferably 8 to 17% by weight, particularly preferably 11 to 16% by weight, exceptionally preferably 12 to 13% by weight of PPG-14 butyl ether, furthermore
c3) 0 to 18% by weight, preferably 0.1 to 15% by weight, particularly preferably 0.5 to 13% by weight, exceptionally preferably 1 to 7% by weight of 2-phenoxyethyl octanoate, and
c4) 0 to 7% by weight, preferably 0.3 to 6% by weight, particularly preferably 1 to 5% by weight, exceptionally preferably 2 to 4% by weight of PPG-3 myristyl ether, and
c5) at least one additional oil, different from the oils c1, c2, c3, and c4, in a total amount of 0.1 to 12% by weight, particularly preferably 0.1 to 11% by weight, exceptionally preferably 3 to 10% by weight,
   with the proviso that the total amount of oils c2)+c3)+c4) is a maximum of 30% by weight and with the proviso that at least one of the oils, 2-phenoxyethyl octanoate or PPG-3 myristyl ether, is included,
   in antiperspirant compositions, including at least one active antiperspirant substance, which is present in suspended, undissolved form and is selected from aluminum salts and aluminum-zirconium salts, and 0-7% by weight, preferably 0-3% by weight of free water, wherein all quantities given in percentage by weight in each case refer to the weight of the composition, without taking into account optionally present propellants,
   for residue masking and/or for reducing the textile staining potential of the composition.

“Reducing the textile staining potential” according to the invention is understood to mean that the composition, tested by the method described below for evaluating spot formation, has a delta L value in the range of −0.9 to +0.9, preferably in the range of −0.7 to +0.7, particularly preferably in the range of −0.7 to +0.4, and exceptionally preferably in the range of −0.7 to 0.

The statements made about the compositions of the invention apply mutatis mutandis in regard to other preferred embodiments of the use of the invention.

### Examples

To prepare the antiperspirant suspensions, the ingredients (see table) were mixed at 30° C. and homogenized. The formulations were filled into aerosol cans in the weight ratio of 1:5.7 with the propellant propane/butane (15/85). The aerosols were sprayed for 2 seconds onto black cardboard and the white residue was determined in triplicate by comparison with a reference scale (0=no white residue, 4=very significant white residue). To evaluate the spot formation, the aerosols were sprayed for 2 seconds onto pale-blue cotton, then incubated for 60 minutes with an electrolyte solution, and washed at 40° C. in a commercial washing machine, dried, and ironed. The procedure was repeated four times. The spots were evident on the pale-blue material as dark areas with a wet appearance. The delta L value (L_{Spot}−L_{Untreated area}) was determined as a measure for the dark spot formation by colorimetric measurement (Minolta/Konica Colorimeter, type: CR400).

Table 1 shows examples of formulations of the invention with a residue formation of a maximum of 1.5 units and staining with a maximum delta L −0.7. (The dark staining on the pale-blue textile is the greater, the more negative the delta L value.)

The comparison formulations in Table 2 show that there is significant spot and/or residue formation except for the parameters established in Section IV.

TABLE 1
Residue and spot formation with formulations of the invention E1
to E4. The listed oils in total always comprise 58% by weight of
the formulations. The rest of the components are 4% by weight of
triethyl citrate, 6% by weight of isopropyl myristate, 5.1% by weight
of perfume oil, 2.5% by weight of disteardimonium hectorite, 23.5%
by weight of aluminum chlorohydrate, and 0.9% by weight of propylene
carbonate. All quantities are given in % by weight.

| | E1 | E2 | E3 | E4 |
|---|---|---|---|---|
| PPG-14 butyl ether | 8 | 17 | 12.7 | 17 |
| Phenoxyethyl caprylate | 15 | 7.1 | 12.3 | / |
| PPG-3 myristyl ether | / | / | 2.7 | 6 |
| Cyclopentasiloxane | 35 | 33.9 | 30.3 | 35 |
| Residue (score) | 1 | 1.5 | 1 | 1.5 |
| Staining (delta L) | 0.4 | −0.7 | −0.1 | −0.5 |

TABLE 2
Residue and spot formation with comparison formulations V1 to V10. The listed oils in total
always comprise 58% by weight of the formulations. The rest of the components are 4% by
weight of triethyl citrate, 6% by weight of isopropyl myristate, 5.1% by weight of perfume oil,
2.5% by weight of disteardimonium hectorite, 23.5% by weight of aluminum chlorohydrate,
and 0.9% by weight of propylene carbonate. All quantities are given in % by weight.
The deviations from the parameters of the invention are highlighted in bold.

| | V1 | V2 | V3 | V4 | V5 | V6 | V7 | V8 | V9 | V10 |
|---|---|---|---|---|---|---|---|---|---|---|
| PPG-14 butyl ether | 11.3 | 5.0 | 17.0 | 8.0 | 11.4 | 17.0 | 15.0 | — | 17.0 | 17.0 |
| Phenoxyethyl | 9.0 | 15.0 | 15.0 | — | 15.0 | 7.4 | 15.0 | 11.5 | 15.0 | — |
| caprylate | | | | | | | | | | |
| PPG-3 myristyl | 9.3 | 7.9 | 15.0 | 15.0 | 11.7 | 15.0 | 2.8 | 11.5 | 2.4 | 15.0 |
| ether | | | | | | | | | | |
| Cyclopentasiloxane | 28.4 | 30.1 | 11.0 | 35.0 | 19.9 | 18.6 | 25.2 | 35.0 | 23.6 | 26.0 |
| Residue (score) | 1.2 | 0.5 | 1.5 | 1.9 | 0.5 | 2.5 | 1.0 | 0.5 | 2.0 | 1.0 |
| Staining (delta L) | −2.2 | −2.2 | −5.9 | −1.0 | −3.9 | −4.8 | −4.3 | −2.8 | −3.0 | −3.3 |

Formulation examples for antiperspirant sticks of the invention

| | 1 | 2 | 3 |
|---|---|---|---|
| | % by | % by | % by |
| | weight | weight | weight |
| Aluminum chlorohydrate (Microdry 3115 | — | 22.0 | — |
| from Summit Reheis) | | | |
| Aluminum-zirconium pentachlorohydrex Gly | 22.0 | — | — |
| (AAZG 3110 from Summit Reheis) | | | |
| Aluminum-zirconium trichlorohydrex Gly | — | — | 17.8 |
| (AAZG 531 from Summit Reheis) | | | |
| PPG-14 butyl ether (Ucon Fluid AP from | 9.5 | 9.5 | 9.5 |
| DOW) | | | |
| 2-Phenoxyethyl octanoate | 7 | 6 | 9.5 |
| PPG-3 myristyl ether | 2.5 | 3.5 | — |
| Talc | — | — | 3.0 |
| Stearyl alcohol, (Lanette 18 from BASF) | 20.0 | 20.0 | 19.0 |
| Cocoglycerides (Novata AB PH from BASF) | — | — | — |
| Hydrogenated castor oil (Cutina HR from | 3.0 | 3.0 | 4.8 |
| BASF) | | | |
| Cyclopentasiloxane | 32.0 | 33.0 | 27.7 |
| Ceteareth 30 (Eumulgin B3 from BASF) | 1.0 | — | 2 |
| Myristyl myristate (Crodamol MM) | 2.0 | 2.0 | 2.0 |
| Cetyl alcohol | — | — | 2.5 |
| Performalene 500 Polyethylene | — | — | 1.2 |
| Perfume | 1.0 | 1.0 | 1.0 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant composition for personal body care, comprising
a) at least one active antiperspirant substance, which is present in suspended, undissolved form and is selected from aluminum salts and aluminum-zirconium salts,
b) 0-7% by weight of free water,
c) a mixture of at least three oils that are liquid under normal conditions,
wherein the composition includes
c1) at least one volatile silicone oil in a total amount of 28 to 61% by weight, furthermore
c2) 7 to 20% by weight by weight of PPG-14 butyl ether, furthermore
c3) 0 to 18% by weight by weight of 2-phenoxyethyl octanoate, and
c4) 0 to 7% by weight by weight of PPG-3 myristyl ether,
with the proviso that the total amount of oils c2)+c3)+c4) is a maximum of 30% by weight, wherein all quantities given in percentage by weight in each case refer to the weight of the composition, without taking into account optionally present propellants, and with the proviso that at least one of the oils, 2-phenoxyethyl octanoate or PPG-3 myristyl ether, is included.

2. The antiperspirant composition of claim 1, wherein the composition comprises 0-3% by weight of free water.

3. The antiperspirant composition of claim 1, wherein the composition comprises 30 to 50% by weight of at least one volatile silicone oil.

4. The antiperspirant composition of claim 1, wherein the composition comprises 32 to 45% by weight of at least one volatile silicone oil.

5. The antiperspirant composition of claim 1, wherein the composition comprises 35 to 38% by weight of at least one volatile silicone oil.

6. The antiperspirant composition of claim 1, wherein the composition comprises 8 to 17% by weight of PPG-14 butyl ether.

7. The antiperspirant composition of claim 1, wherein the composition comprises 12 to 13% by weight of PPG-14 butyl ether.

8. The antiperspirant composition of claim 1, wherein the composition comprises 0.1 to 15% by weight of 2-phenoxyethyl octanoate.

9. The antiperspirant composition of claim 1, wherein the composition comprises 1 to 7% by weight of 2-phenoxyethyl octanoate.

10. The antiperspirant composition of claim 1, wherein the composition comprises 0.3 to 6% by weight of PPG-3 myristyl ether.

11. The antiperspirant composition of claim 1, wherein the composition comprises 2 to 4% by weight of PPG-3 myristyl ether.

12. The composition according to claim 1, wherein the at least one volatile silicone oil is selected from octamethylcyclotetrasiloxane, decamethylcyclopentasiloxane, dodecamethylcyclohexasiloxane, hexamethyldisiloxane, octamethyltrisiloxane, and decamethyltetrasiloxane, and mixtures thereof, in particular decamethylcyclopentasiloxane, mixtures of decamethylcyclopentasiloxane and dodecamethylcyclohexasiloxane, and mixtures of hexamethyldisiloxane, octamethyltrisiloxane, and decamethyltetrasiloxane.

13. The composition according to claim 1, wherein the at least one additional oil, different from the oils c1, c2, c3, and c4, is included in a total amount of 0.1 to 12% by weight of the composition, without taking into account optionally present propellants.

14. The composition according to claim 1, wherein the at least one additional oil, different from the oils c1, c2, c3, and c4, is included in a total amount of 3 to 10% by weight of the composition, without taking into account optionally present propellants.

15. The composition according to claim 1, wherein at least one additional oil, different from the oils c1, c2, c3, and c4, is present in the composition, and which at least one additional oil is selected from the group consisting of: esters of linear or branched, saturated or unsaturated fatty alcohols having 2 to 30 carbon atoms with linear or branched, saturated or unsaturated fatty acids having 2 to 30 carbon atoms, which may be hydroxylated, from the mono- and polyesters of lactic acid, citric acid, tartaric acid, or adipic acid with a monohydric alcohol having 2 to 9 carbon atoms, from the dicarboxylic acid esters of linear or branched C₂-C₁₀ alkanols, of the benzoic acid esters of linear or branched C₈₋₂₂ alkanols, from the mono- and polyesters of lactic acid, citric acid, tartaric acid, or adipic acid with a dihydric, trihydric, or tetrahydric alcohol having 2 to 9 carbon atoms, and mixtures thereof.

16. The composition according claim 15, wherein at least one additional oil is present in an amount of 0.1 to 11% by weight, based on the weight of the composition without taking into account optionally present propellants, and is selected from the group consisting of: isopropyl myristate, isopropyl palmitate, isopropyl stearate, 2-ethylhexyl palmitate, 2-ethylhexyl stearate, and triethyl citrate, and mixtures thereof.

17. The composition according to claim 1, wherein the at least one active antiperspirant substance is present in the composition in a total amount of 5-40% by weight, based on the total weight of the active substance (USP), free from water of crystallization, in the composition, without taking into account optionally present propellants.

18. The composition according to claim 1, wherein the at least one fragrance is present in the composition in a total amount of 0.1-15% by weight, based in each case on the total weight of the propellant-free composition.

19. The composition according to claim 1, wherein the at least one thickener comprises 0.5-10% by weight, based on the total weight of the propellant-free composition, and is a hydrophobized clay mineral.

20. The composition according to claim 1, wherein the at least one antiperspirant active substance is present in the composition in a total amount of 5-40% by weight based on the total weight of the active substance (USP), free from water of crystallization, in the composition, without taking into account optionally present propellants.
